(19) United States
(12) Patent Application Publication
(10) Pub. No.: US 2026/0198988 A1
(43) Pub. Date: Jul. 16, 2026

(54) GENERATOR FOR OPERATING SURGICAL INSTRUMENTS

(71) Applicant: Erbe Elektromedizin GmbH, Tuebingen (DE)

(72) Inventors: Thomas EGLER, Reutlingen (DE); Thomas RIPPLINGER, Gomaringen (DE)

(21) Appl. No.: 19/443,329

(22) Filed: Jan. 8, 2026

(30) Foreign Application Priority Data
Jan. 10, 2025 (EP) 25151151.5

(51) Int. Cl. A61B 18/12; A61B 18/00

(52) U.S. Cl. CPC A61B 18/1206; A61B 2018/00642; A61B 2018/00678; A61B 2018/00702; A61B 2018/00892; A61B 2018/1253; A61B 2018/1286

(57) ABSTRACT

A generator suitable for the pulsed operation comprises a power factor correction circuit having an integrated circuit. The voltage detector input of the control circuit is upstream of and connected to an amplifier. The amplifier may be configured so that its amplification factor has a value of 1 in a first condition and takes a value larger than 1 in a second condition. Moreover, the amplification may be set to a value of larger than 1 only if the voltage at the converter output is inside a tolerance range that is provided for the usual operation of the power factor correction circuit. Outside of this tolerance range, the amplification of the amplifier is then exactly 1. In doing so, the usual operation of the integrated control circuit is not disturbed, particularly during start-up.

### CROSS REFERENCE TO RELATED APPLICATION

This application claims the benefit of European Patent Application No. 25151151.5, filed Jan. 10, 2025, which is incorporated herein by reference in its entirety.

### TECHNICAL FIELD

The invention relates to a generator for operating one or more surgical instruments, particularly for supply of such instruments with electrical power.

### BACKGROUND

Electrosurgical instruments for use on human or animal patients are in general known from the prior art, just as generators for supply of such instruments. In this regard EP 2 853 217 B1 discloses a generator to which a monopolar instrument as well as an associated neutral electrode are connected. For energy supply the generator can be connected to the public current grid.

The generator comprises a grid rectifier at its input side to which a power factor correction circuit is connected downstream. The latter is configured as a boost converter and charges a storage capacitor up to a voltage that is above the peak voltage of the highest considerable grid voltage. Thereby the storage capacitor has the task to store the required energy in order to avoid a too large voltage drop, also in case of pulsating loads, at least a voltage drop below the grid peak voltage, so that no uncontrolled current flow occurs through the grid rectifier and the boost converter to the storage capacitor. A DC voltage converter is connected to the boost converter, wherein the DC voltage converter has an inverter and a transformer for reliable potential separation between the patient side electrical equipment and the grid side electrics. The DC voltage converter as well comprises controlled switches and buffer capacitors. The power factor correction circuit as well as the DC voltage converter comprise controls that communicate among each other via a data interface. Connected to the DC voltage converter is a radio frequency oscillator, which provides the radio frequency treatment voltage required for supply of a surgical instrument.

For realization of power factor correction circuits integrated circuits are available, such as ICE3PCS01-DS from Infineon Technologies, the characteristics of which and application recommendations are apparent and available from www.infineon.com.

In the case of varying loads, voltage fluctuations occur on the storage capacitor of the power factor correction circuit, the limitation of which requires a respectively large scale dimensioning of the storage capacitor. Resulting therefrom are remarkable space requirements and also error susceptibilities resulting from the charging and discharging current stress of the buffer capacitor or a respective capacitor block.

### SUMMARY

Starting therefrom it is one object of the invention to provide a generator having a power factor correction circuit comprising a reduced assembly space and an increased reliability.

This object is solved by means of a generator as described herein.

The generator according to the invention serves for operation of surgical instruments, that means for supply thereof with typically radio frequency voltage and radio frequency current, wherein the operation of the instruments can be pulsed. The pulsing can already be created in that the instrument is repeatedly switched on and off again for seconds as it is carried out by the surgeon in the context of his/her surgery. The pulsing can also result from the fact that the mode selected for operation of the instrument requires a continuous on and off switching of the voltage of the RF generator. This pulsation can be in the sub-Hertz range or also in the range of one or a few Hertz. Pulsations of higher frequency are also possible.

As it is common, the power factor correction circuit is based on a boost converter circuit, the input of which is connected with a grid rectifier and the converter output of which is connected with at least one storage capacitor. In addition, the boost converter comprises an electronic switch having a control electrode, for example a field effect transistor having a gate electrode. For a control of the electronic switch a control circuit is provided, whose switching signal output is connected with the control electrode, for example the gate of the field effect transistor. In addition, the control circuit comprises a voltage detector input, which is, for example, connected with the converter output via a voltage tap circuit. In this manner the control circuit receives a signal at its voltage detector input which characterizes the voltage present on the storage capacitor.

The control circuit is typically an integrated circuit that is configured for the operation of power factor correction circuits, commercially produced and distributed in large scale and is thus simply available on the market. Typically, such circuits are, however, not suitable for power factor correction circuits that supply abruptly and considerably varying loads, particularly slowly pulsating loads in the Hertz or sub-Hertz range, or that would require excessively large capacitor packets for this purpose. The invention provides remedy for this in that a signal amplifier is arranged between the voltage tap circuit and the voltage detector input of the control circuit. Thereby the control circuit becomes suitable for the operation of power factor correction circuits that can supply considerably varying loads, particularly also pulsed loads, without internal intervention.

The control circuit is preferably an integrated circuit, for example, an ICE3PCS01G from Infineon Technologies, an L4985 from the manufacturer STMicroelectronics, a TEA2376DT from the manufacturer NXP or a UCC28180 from Texas Instruments. Additional ICs from these or other manufacturers can also be used.

The circuits are designed and typecasted according to their standard applications for operation with uniform or gradually changing load. They are also suitable for quickly changing loads, whereby, however, temporary voltage fluctuations at the converter output have to be expected in the case of quick load changes. Due to the additional amplifier provided according to the invention and connected upstream of the voltage detector input, the power factor correction circuit becomes suitable also for operation of quickly changing loads while concurrently avoiding larger voltage fluctuations, so that the generator can also provide modes with a pulsed operation using this power factor correction circuit. This is possible without the need to counteract temporary voltage deviations with an enlarged storage capacitor (packet).

Control circuits of the indicated configuration can have an overvoltage switch-off function that is configured to switch off the boost converter if the voltage at the voltage detector input VSENSE exceeds a threshold. Additionally, the control circuit can comprise another signal input OVP that is connected with the converter output via a voltage divider circuit as necessary, in order to monitor the converter output for overvoltage. In order to not supply inconsistent signals to the voltage detector input VSENSE and the additional signal input OVP during start-up of the boost converter after start with empty storage capacitor, that means during switch-on in case of still uncharged storage capacitor, it is expedient to be able to vary the amplification factor of the amplifier during the operation of the power factor correction circuit. In doing so, it can be avoided that the control circuit switches into an error mode and switches off the power factor correction circuit.

Particularly, the amplifier can comprise a control input configured for control of the amplification. The amplification factor of the amplifier can be switched between at least two different values via the control input. Preferably, the first value is equal to one and the second value is larger than one. This concept is particularly suitable for ICs in which the amplification of the signal supplied to the voltage detector input VSENSE is not provided. In such ICs otherwise inconsistent signals can result at the different inputs of the ICs due to the additional signal amplification. This can result in difficulties, such as error switch-off, for example during start-up of the circuit, particularly during cold boot.

In a preferred embodiment the control circuit comprises a signal output VB_OK that characterizes that the desired set point voltage is provided at the converter output of the power factor correction circuit. This signal output VB_OK is preferably connected with the switching input of the amplifier. Thereby it is achieved that the power factor correction circuit receives an unamplified signal at its voltage detector input after switching on and thus controls the boost converter according to its specification. If the set point voltage at the converter output is achieved, for example 400 Volt, the signal provided at the signal output VB_OK of the control circuit changes its value. This signal is supplied as switching signal to the control input of the amplifier, whereby the latter now comprises an amplification factor that is then larger than one. In doing so, the loop amplification in a control loop formed by the amplifier and the control circuit is increased, which now results in an improved control accuracy once the set point voltage (for example 400 Volts) has been reached. In so doing, it becomes possible to downsize the capacitor or the capacitor packet provided for buffering load fluctuations at the converter output, which saves installation space and also allows an increase of the reliability of the generator in total, because of reduced charging and discharging currents. On the other hand, an interference of the operation of the IC, particularly during the start-up phase, is avoided.

### BRIEF DESCRIPTION OF THE DRAWINGS

Further details and advantages of the present invention are derived from the drawing as well as the description and the associated figures as well as the claims. The drawings show:

FIG. 1 a generator for operating surgical instruments with the illustration of its function blocks,

FIG. 2 a grid rectifier and the power factor correction circuit PFC in addition to the control circuit of the generator according to FIG. 1 in form of a basic circuit diagram,

FIG. 3 the controllable amplifier of the power factor correction circuit according to FIG. 2 in form of a basic circuit diagram,

FIG. 4 a circuit branch of the controllable amplifier according to FIG. 3,

FIG. 5 a switch-on delay circuit being part of the power factor correction circuit according to FIG. 2, and

FIG. 6 a diagram for illustration of the start-up operation of the pulsed operation of the power factor correction circuit.

### DETAILED DESCRIPTION

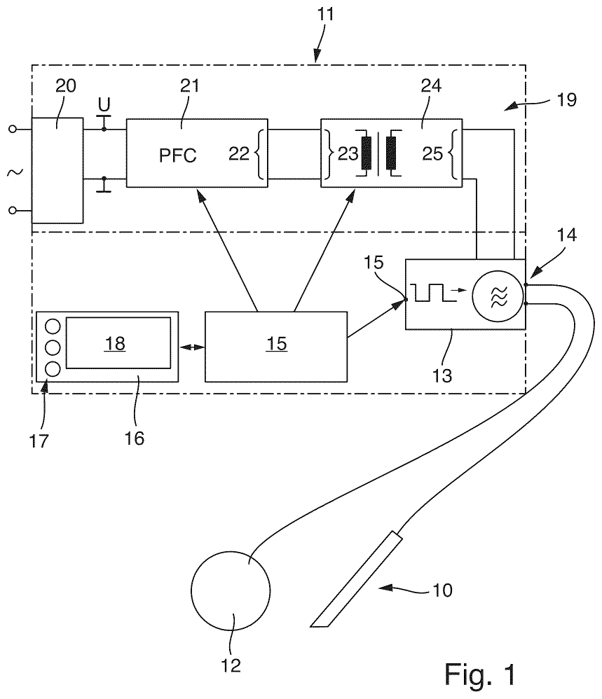

In FIG. 1 a medical instrument 10 for surgical or other effects on a patient and a generator 11 serving for supply of the instrument 10 are illustrated. The instrument 10 is illustrated as monopolar instrument to which a neutral electrode 12 is assigned which has to be attached to the patient. The neutral electrode 12 and the instrument 10 are connected with generator 11 via electrical lines. Instead of a monopolar instrument, however also bipolar or multipolar instruments can be used, which then do without a neutral electrode where applicable.

The generator 11 is particularly configured and suitable to operate instrument in modes in which the electrical load provided by the instrument and thus the electrical power consumed by the instrument abruptly changes between very low values and very high values. The low values can be values close to zero Watt or of only a few Watts of electrical power. The high values can be powers of multiple 100 Watts up into the Kilowatt range.

The generator 11 comprises a radio frequency oscillator 13 configured to provide the required electrical power at an output 14 to which the instrument 10 and the neutral electrode 12 are connected. In addition, the radio frequency oscillator 13 comprises a control input 15, which is configured to receive control impulses that control the radio frequency oscillator 13. For example, the control impulses can effect switch-on and switch-off of the radio frequency oscillator or also another power modulation thereof. In FIG. 1 as an example for the temporal progress of a control impulse a square wave is illustrated inside the block characterizing the radio frequency oscillator 13, according to which the radio frequency oscillator 13 is switched on and off in defined time intervals. Thereby the intervals between the switch-on and switch-off points in time, that means between the front and the back flank of an impulse of a square wave, can have an amount of some 10 to some 100 milliseconds or also one or multiple seconds. In other words, the frequency of these control impulses can be in the sub-Hertz or in the Hertz range.

A system control 15 serves for the creation of the control impulses and thus for defining the mode in which the entire generator 11 and particularly the radio frequency oscillator 13 operate. The system control is connected with a communication unit 16, which is configured to receive user inputs and to indicate outputs. For this purpose, the communication unit 16 comprises input element 17, for example in form of keys, buttons, or switches, as well as an output unit 18, for example in form of one or more screens and/or indicator instruments and/or control lamps or the like.

For current supply, particularly of the radio frequency oscillator 13, but also the system control 15 and the communication unit 16, serves a current supply unit 19 illustrated in the upper part of generator 11. The current supply unit 19 is connected to the public current supply grid on its input side and supplies the components of generator 11 with current in potentially isolated manner with requisite electrical reliability. This means that the current supply unit 19 is configured in a potential isolating manner between the grid side and the patient side, so that potential differences of multiple thousand Volts between the current supply grid on one side and the patient or the instrument 10 and the neutral electrode on the other side do not result in a harmful electrical current flow through the patient.

First, an input rectifier 20 having a grid filter connected to the electrical grid is part of the current supply unit 19. The input rectifier 20 is typically configured as bridge rectifier and supplies a rippled direct voltage Ur at its output, which is supplied to an input of the power factor correction circuit 21. The power factor correction circuit 21 (PFC) transforms this voltage in a direct voltage applied at its output, which is higher than the peak voltage of the grid voltage.

The power factor correction circuit 21 comprises an output 22, where the direct voltage is provided that has been transformed by the power factor correction circuit 21. The output 22 is connected to an input 23 of a potential isolating voltage converter 24, the output 25 of which is in turn connected with the radio frequency oscillator 13 in order to supply the latter with electrical power. The voltage converter 24 is configured in potential isolating manner, that means the input 23 and the output 25 are galvanically separated. The dielectric strength of this galvanic insulation is typically in the range of above 6 kV, better 10 kV or 12 kV.

Optionally a controlling connection, for example in form of a data connection, can be provided between the system control 15 and the voltage converter 24. For example, this data connection can serve to set the amount of the voltage output at the output 25 or other parameters. Likewise, as an option, a controlling connection between the system control 15 and the power factor correction circuit 21 can be provided, for example in order to activate or deactivate the power factor correction circuit 21, for example in order to preset a standby mode.

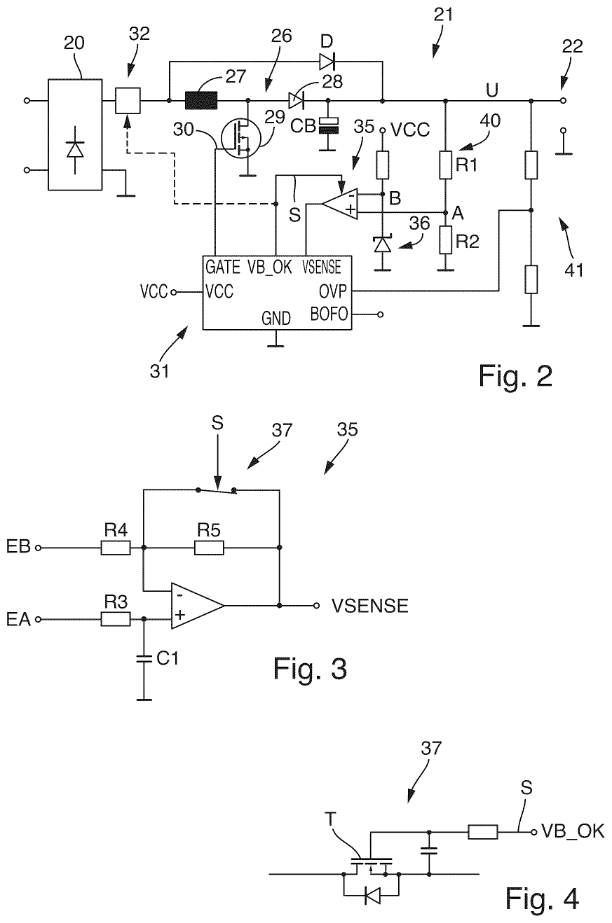

A main focus of the invention is the configuration of the power factor correction circuit 21, which is represented in FIG. 2 in form of a basic circuit diagram. The power factor correction circuit 21 comprises a boost converter circuit 26, the main components of which are an inductor 27, a diode 28 connected thereto in series in flow direction, an electronic switch 29 leading from a point therebetween to ground and a storage capacitor CB connected to the diode and to the ground. The controllable switch 29 is preferably a field effect transistor, the source of which is connected to ground and the drain of which is connected with the connection point between the inductor 27 and the diode 28. Its control electrode 30 (in the case of a field effect transistor its gate) is connected with a control circuit 31 that is configured as integrated circuit. A preferred component for the controlled circuit 31 is the circuit ICE3PCS01G from the manufacturer Infineon Technologies. In the market additional suitable integrated circuits are provided that can be used here, for example L4985 of ST Microelectronics, TEA2376DT from NXP and UCC28180 from TXP and many others more.

The boost converter circuit 26 largely corresponds to the standard circuit, which can be taken from the datasheet of the control circuit 31, apart from the particularities described in the following. Connections of the control circuit 31 and their external wiring and connection that are not necessary for the comprehension of the circuit have been omitted in FIG. 2. The connections are nevertheless available and can be wired/connected as indicated in the datasheet.

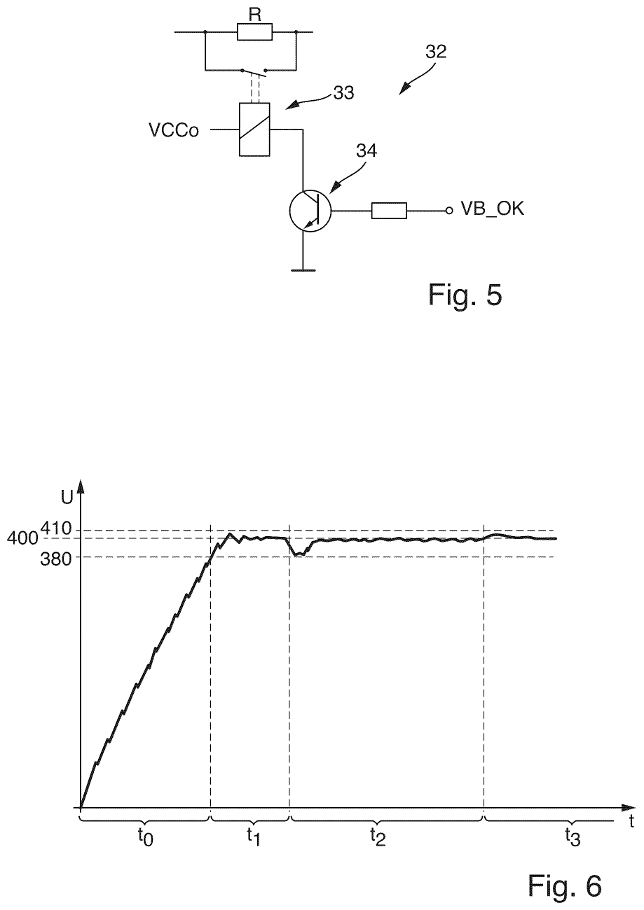

Between the input rectifier 20 and the boost converter circuit 26 an inrush current limiting circuit 32 is provided which is separately illustrated in FIG. 5. In the end the inrush current limiting circuit 32 is a current limiting resistor R which is short-circuited by the switching contact of a relay 33, as soon as a respective connection VB_OK of control circuit 21 changes to a positive potential different from zero in order to make the connected transistor 34 conductive and thereby close the contact of relay 33. A positive voltage different from zero is applied to the connection VB_OK as soon as the voltage at the converter output 22 reaches a set point range. The latter is in the present case according to the dimensioning of a voltage tap circuit 40 between 380 and 410 V, in case of a desired converter output voltage U of 400 V. The voltage tap circuit 40 is a voltage divider circuit having two or more ohmic resistors R1, R2.

The control circuit 31 additionally comprises an overvoltage protection input OVP which is connected to the converter output 22 via a voltage divider 41. The voltage divider is thereby dimensioned so that a switch-off limit at the overvoltage protection input OVP is only reached if a non-acceptable overvoltage has been determined at the converter output 22. For example, the latter is defined by the dielectric strength of the storage capacitor CB and can have an amount of 420 V, for example.

The particularity of the boost converter circuit 26 compared with a standard use of control circuit 31 is that an amplifier is arranged between the voltage detector input VSENSE and the voltage tap circuit 40. The amplifier 35 comprises a non-inverting input, which is connected with the voltage tap point A of the voltage tap circuit 40 consisting of the resistors R1 and R2. The amplifier output is conversely connected with the voltage detector input VSENSE.

In a preferred embodiment the amplifier 35 additionally comprises an inverting input which is connected to a reference voltage. The latter can be created at a voltage standard, for example in form of a reference voltage source 36, for example a Zener-diode via a series resistor from the supply voltage VCC (for example 12V). The reference voltage source 36 is configured to provide the voltage, which also applies at the voltage tap point A, if the voltage U is at its set point value. In this case the difference between the voltage at the voltage tap point A and the reference voltage is equal to zero. In addition, the reference voltage is equal to the voltage that has to be applied to the input VSENSE of the control circuit (particularly ICE3PCS01G from Infineon), if the voltage U at the output 22 is equal to its set point value. This voltage of the reference voltage source 36 VSENSE neither results to an increase nor to a reduction of the voltage U if it is applied to the input VSENSE. In the present embodiment a reference voltage source of 2.5 V is used, for example in form of ADR5041BKSZ of Analog Devices Inc.

The amplifier 35 can be configured as amplifier with switchable amplification factor as illustrated in FIG. 3. The amplifier 35 can be an operational amplifier, the non-inverting input of which is connected with the amplifier input EA via a resistor R3. The inverting input is conversely connected with the amplifier input EB via a resistor R4, the dimension of which is equal to the dimension of resistor R3. In the feedback branch between the output of the operational amplifier and the resistor R4 a resistor R5 is arranged, the ratio of which in relation to the resistor R4 determines the amount of the amplification factor. In parallel to resistor R5 a switch 37, particularly an electronic switch, can be provided, by means of which the resistor R5 can be short-circuited. A signal S can serve for control of switch 37 by means of which switch 37 can be specifically opened or closed.

FIG. 4 illustrates a realization of switch 37 by means of a field effect transistor T, the gate of which is connected to the connection VB_OK of control circuit 31 via a resistor.

The amplification factor of amplifier 35 is “one” in closed condition, that means current conducting switch 37. On the contrary if switch 37 is opened, that means current blocking, the amplification factor is determined by the ratio of the resistors R4 and R5 in relation to one another. If these two resistors have equal amounts, the amplification factor is “two” as it is preferred in the present case. However, it is also possible to provide other amplification factors.

The generator 11 described so far operates as follows:

After switching the generator on, the power factor correction circuit 21 has to charge the storage capacitor CB first. For this purpose, a charging current can flow via diode D, wherein the charging current is at first limited by means of the inrush current limiting circuit 32. The signal S at the connection VB_OK is zero, whereby it is indicated that the voltage at the converter output 22 is still outside of the setpoint voltage range. Because switch 37 is closed in this condition, the amplifier 35 has the amplification equal to one. In other words, at the input VSENSE of control circuit 31 the reference voltage of the reference voltage source 36, which is-depending on the sign-increased or decreased about the difference between the voltage at the voltage tap point A and the reference voltage. The boost converter circuit 26 thus operates in a common manner with the control amplification internally set by control circuit 31 in the time phase t0 indicated in FIG. 6.

As soon as the voltage applied to the storage capacitor CB and thus also to the converter output 22 reaches a lower limit value of a voltage tolerance range, the output VB_OK that indicates the reaching of the setpoint voltage changes to a positive value different from zero. Due to suitable dimensioning of the voltage tap circuit 40 formed by a voltage divider or the voltage divider 41 at the connection OVP, this limit value can be appropriately defined, for example to 380 Volts.

In that signal S switches to a positive value upon reaching the preset switching threshold defined in this manner, on one hand the inrush current limitation 32 is deactivated due to the short circuit of resistor R and on the other hand the switch 37 is opened. Thereby, now, the reference voltage is increased or decreased about the two times amplified difference between the voltages at the voltage tap points A and B (FIG. 2) and is provided to the voltage detector input VSENSE. The voltage feedback control of control circuit 31 and the control loop formed by the latter now operates with increased amplification.

This is the case as soon as and as long as the voltage U at the converter output is inside a defined tolerance range, such as between 380 and 410 Volts according to FIG. 6. If the control circuit 31 formed by an integrated circuit is designed as proportional controller (P-controller), proportional-integral controller (PI-controller) or proportional-integral-derivative controller (PID-controller), the proportional component P of the controller is at least increased by the invention if (and preferably only if) the voltage U at the output 22 is inside a predefined tolerance range. This can be indicated by means of a signal at an output VB_OK of the integrated control circuit 31.

With the increased amplification of amplifier 35 now the desired setpoint voltage of 400 Volts is controlled in feedback manner, wherein, for example, temporary deviations due to abrupt load changes are minimized by means of the increased amplification. This is illustrated by a comparison of the time phases t1, t2 and t3 in FIG. 6 following the switch-on. In the time phase t1 low current is consumed at the converter output 22. After the built-up transient phase of the voltage onto the setpoint voltage of 400 Volts the voltage remains constant. At the beginning of the subsequent time phase t2, which can have an amount of 100 ms, 200 ms or multiple 100 ms, for example, high power is consumed at the converter output 22, for example maximum power. The transition between low and high power can be very quick, for example during only a few milliseconds or in a fraction thereof. Thereby the voltage drops down during a short time phase, in order to being subsequently controlled very quickly again onto the setpoint value of 400 Volts. The visible low remaining ripples result from the grid ripples, but not from control effects.

Due to the amplification of the signal taken from the voltage tap points A and B, the voltage drop of voltage U at the output 22 is significantly lower than it would be without this additional amplification with equally dimensioned storage capacitor CB. Also, the voltage increase after the end of the load period t2 is significantly lower than it would be without amplifier 35. Therefore, using the power factor correction circuit 21 described so far, generators 11 can be realized whose radio frequency oscillator 13 comprise operating periods that change very strongly and in short term even between zero and full load without the need to dimension the storage capacitor CB larger than usual. The capacitor CB can be dimensioned only to the value that it would need for compliance with the requested remaining ripples and the requested full load without consideration of abrupt load changes considered here.

The amplifier 35 operates with an amplification larger than one as soon as and as long as the signal at the connection VB_OK is different to zero, that means as soon as and as long as the voltage U at the converter output 22 is inside the desired tolerance range. Outside of this tolerance range the amplifier 35 operates with an amplification factor equal to one, that means without amplification.

A generator 11 suitable for the pulsed operation according to the invention comprises a power factor correction circuit 21 having an integrated control circuit 32. The latter comprises a feedback path via which a control loop is formed. The power factor correction circuit 21 is configured to control the switch 29 by means of switching impulses so that voltage fluctuations at the voltage detector input VSENSE are counteracted.

The amplifier 35 is arranged in the feedback path provided for voltage control, wherein the amplifier comprises an amplification factor larger than 1. For this purpose, the voltage detector input (VSENSE) of control circuit 31 is connected to the amplifier 35 upstream. In doing so, voltage fluctuations at the converter output 22 can be minimized, which occur as a consequence of abrupt load changes. It is possible to configure the amplifier 35 so that its amplification factor has the value 1 (or another unchangeable value) in a first condition and a value deviating therefrom, preferably a larger value, in a second condition. Moreover, it is possible to set the amplification to a value of larger than 1 only if the voltage at the converter output is inside a tolerance range which is provided for the normal operation of the power factor correction circuit 21. Outside this tolerance range, the amplification of amplifier 35 is then exactly 1. Thereby the normal operation of the integrated control circuit 31 is not disturbed, particularly during start-up.

### REFERENCE SIGNS

10 instrument
11 generator
12 neutral electrode
13 radio frequency oscillator
14 output
15 system control
16 communication unit
17 input elements
18 indicator elements
19 current supply unit
20 input rectifier
Ur voltage at the output of input rectifier 20
21 power factor correction circuit
22 converter output
23 input of voltage converter 24
24 voltage converter
25 output
26 boost converter circuit
27 inductor
28 diode
29 controllable switch/transistor
30 control electrode
31 control circuit
32 inrush current limiting circuit
R current limiting resistor
33 relay
VB_OK connection for indication of set point voltage
34 transistor
U converter output voltage
OVP overvoltage protection input
VSENSE voltage detector input
35 amplifier
36 reference voltage source
VCC supply voltage
37 switch
T field effect transistor
40 voltage tap circuit
41 voltage divider

1. A generator (11) for operation of surgical instruments (10), the generator comprising:
a power factor correction circuit (21) comprising a boost converter circuit (26), the boost converter circuit comprising an input which is connected to a grid rectifier (20) and a converter output (22) which is connected with at least one storage capacitor (CB);
the power factor correction circuit (21) further comprising an electronic switch (29) having a control electrode (30);
a voltage tap circuit (40) connected to the converter output (22) and comprising a voltage tap point (A);
a control circuit (31) comprising a switching signal output (GATE) connected with the control electrode (30) and a voltage detector input (VSENSE), configured for receiving a signal characterizing a voltage provided to the at least one storage capacitor (CB); and
an amplifier (35) comprising an amplifier input (EA) connected with the voltage tap point (A) and an amplifier output connected with the voltage detector input (VSENSE) of the control circuit (31).

2. The generator according to claim 1, wherein the control circuit (31) is an integrated control circuit that comprises an internally defined relation between the signal at the voltage detector input (VSENSE) and the switching signal output therefrom at the switching signal output (GATE).

3. The generator according to claim 2, wherein the integrated control circuit is configured for application purposes with non-pulsed loads.

4. The generator according to claim 1, wherein the control circuit (31) comprises a signal output (VB_OK) that is configured to output a signal that indicates whether a voltage (U) measurable at the converter output (22) is inside a defined tolerance range.

5. The generator according to claim 1, wherein the control circuit (31) comprises a signal input (OVP) which is configured for detection of an overvoltage at the converter output (22).

6. The generator according to claim 5, wherein the control circuit (31) is configured to compare voltages applied to the voltage detector input (VSENSE) and the signal input (OVP) and to switch off the boost converter circuit (26) if a difference between the voltages exceeds a threshold.

7. The generator according to claim 1, wherein the amplifier (35) is a differential amplifier having an inverting input connected to a reference voltage source (36) and a non-inverting input connected to the voltage tap point (A).

8. The generator according to claim 1, wherein the amplifier (35) comprises an input (S) configured for controlling an amplification of the amplifier.

9. The generator according to claim 8, wherein the input (S) is a switching input that is configured to receive a switching signal in for switching an amplification factor of the amplifier (35) between a first value and a second value.

10. The generator according to claim 2, wherein the first value is equal to one and the second value is larger than one.
